(19) United States
(12) Patent Application Publication
(10) Pub. No.: US 2024/0416058 A1
(43) Pub. Date: Dec. 19, 2024

(54) SYSTEM AND METHODS FOR DYNAMICALLY CONTROLLING OPERATION OF A MECHANICAL VENTILATOR FOR AUTOMATIC TARGETING OF A MONITORED PARAMETER

(71) Applicant: Breas Medical AB, Molnlycke (SE)

(72) Inventors: William A. Truschel, Oakmont, PA (US); Harout Rafi Stepanian, Molnlycke (SE)

(73) Assignee: Breas Medical AB, Molnlycke (SE)

(21) Appl. No.: 18/741,612

(22) Filed: Jun. 12, 2024

Related U.S. Application Data
(60) Provisional application No. 63/521,577, filed on Jun. 16, 2023.

(51) Int. Cl. A61M 16/00; A61M 16/06; A61M 16/08; G16H 40/63

(52) U.S. Cl. CPC A61M 16/024; A61M 16/0003; A61M 16/0066; G16H 40/63; A61M 2016/0027; A61M 16/06; A61M 16/0875; A61M 2202/0007; A61M 2202/0208; A61M 2205/10; A61M 2205/3303; A61M 2205/3327; A61M 2205/3331; A61M 2205/502; A61M 2205/75; A61M 2210/1039; A61M 2230/005; A61M 2230/04; A61M 2230/20; A61M 2230/42

(57) ABSTRACT

A system and method are provided for dynamically controlling operation of a mechanical ventilator for automatic targeting of a monitored parameter. A ventilator prescription includes an initial ventilator configuration, a prescribed target biometric value, comparator formulae for comparing a current condition value to the prescribed target biometric value, and modification formulae for generating modification parameters to modify the current configuration of the system. The ventilator system operates according to the initial configuration, continuously monitors patient biometric sensors and system biomarker calculators, periodically determines a current condition value, and compares the current condition value to the target biometric value. If the current condition value does not meet the target biometric value, the system generates modification parameters to modify the current configuration of the system and automatically reconfigures the current configuration according to the modification parameters to meet the prescribed target value.

### CROSS-REFERENCE TO RELATED APPLICATIONS

This application claims the benefit of U.S. Provisional Patent Application Ser. No. 63/521,577 filed Jun. 16, 2023, the entire contents of which are incorporated herein by reference.

### BACKGROUND OF THE DISCLOSURE

(1) Field of the Invention: The instant invention relates to mechanical ventilator systems, and more particularly to dynamic control of a mechanical ventilator which is not based on algorithmic analysis.

(2) Description of Related Art: The state of the art in the practice of mechanical ventilation is performed under close supervision of a medical care professional in the intensive care unit (ICU). In the ICU, the patient is closely and continuously monitored with external biometric sensors, and the ventilator's alarm system. Any alert provokes an immediate response by the medical professional nearby and that caregiver can assess the patient, make any necessary changes to the therapy or therapy settings and watch, and wait for a positive response and healthy outcome for the patient before a medical emergency develops.

In contrast, when a patient is diagnosed with chronic respiratory insufficiency or chronic respiratory failure, the patient is often sent home with a mechanical ventilator on a fixed prescription. Although biometric sensors and the ventilator alarm system is still active in the home, it is common that no medical professional is nearby to change therapy or change the therapy settings to invoke or initiate a more effective care regimen for the patient. Follow up appointments are typically 6 to 8 weeks after discharge from the care of the intensivists in a hospital or clinic and regularly every 6 to 12 months after the first follow up.

Recent developments in connected care, allow a caregiver to review a patient remotely with a digital dashboard, but this review is often sporadic and spaced days, weeks, or months apart. When we consider that the solution involves allowing the clinician to change the settings for a critical patient, we incur a higher risk, and these remote unsupervised changes are discouraged. In fact, the message from the global regulatory bodies to allow a change to therapy for critical patients when the caregiver is remote is, “don't do it.”

Furthermore, the remote settings solution is not scalable as it requires the constant attention of expert manpower to monitor a large population of homecare patients within a health care system. Due to these challenges, it is expected that patients who need critical attention to a respiratory abnormality will not thrive under the current state of the art.

Due to the nature of the respiratory disease of the patient that may be considered progressive or in the category of a relapsing/remitting type from critical to stable at unknown frequency, it is often likely that therapy setting changes will lag behind the needs of the patient when the intervals of remote monitoring and patient follow-ups are considered.

### SUMMARY OF THE DISCLOSURE

The present disclosure relates to a problem associated with titrating a setting to meet the patient's needs and it addresses the need to regularly update the titration as the patient's needs change. As the patient's disease progresses or instances when the patient relapses into an acute situation or when the patient experiences a temporary change in physiology related to body position, immunological overload, infection, hypersecretion in the airway or other condition, it is often necessary to make a permanent or temporary modification to the prescription to normalize the patient's breathing. The invention allows the clinician to program the device to update and maintain a prescription automatically by allowing the machine to change the prescription while targeting a predetermined measure of normality defined by the clinician.

The process of initially titrating a prescription is often laborious and a burden to skilled personnel. Updating a prescription is also laborious and often ill-timed because patients with chronic disease in the home are often physically and temporally separate from their physicians. Updates are only prescribed during a follow up appointment and only after the laborious task of reviewing the patient's history made visible by downloading stored biometric data. This invention streamlines the process by automatically performing the actions that would be taken at follow up, by reviewing patient data stored on the device or in the cloud, comparing the stored data to a threshold determined to indicate normality and then lastly by automatically adjusting the prescription to achieve the measure normality. This will become clear by illustrating the new process by which the clinician can program a titration strategy into the prescription of one or more settings in the prescription mode.

Titration of a setting is done manually by starting with a typical prescription for the patient's demographics and diagnosis and then evaluating and reevaluating the patient as titration steps are carried out until a condition is satisfied. An example of this is to titrate a pressure support level for a hypercapnic patient to achieve a normal CO₂ level in arterial blood. Different pressure support levels are tried, blood samples are taken and then examined with an analyzer to determine if the pressure support is appropriate to normalize CO₂. When the titration has been performed, it is assumed that this prescription will be suitable across time between doctor visits or remote data examinations, when a modification to the prescription may seem necessary. Furthermore, the titration may be temporarily substandard when the patient undergoes a temporary change in physiology due to an infection or when the patient's metabolic load increases that may require an increase in respiratory support.

Programmable targeting is a system that allows one or more settings within a patient prescription to automatically adjust within a range of settings to target a quantitative value determined by a monitored parameter.

The invention intends to overcome the issues of inappropriate response time incurred due to remote monitoring and more specifically no response when caregivers are absent, or the patient is unable to intervene on his own. The clinician ultimately knowing what is best for an individual or class of patients and has full authority over the intervention actions. No hidden algorithm is contained within the device to automatically make a clinical assessment of the patient and the device does not decide what any appropriate response could be. The device will only operate according to the direction of the programming clinician and there is full transparency of how, when, and why a setting will change. The method described here can be accomplished without using closed loop control as defined by MDR classification rules explicitly mentioned in MDCG 2021-24 under Rule 12.

The key distinction with “programmable ventilation” as described herein, is that control of the ventilator's settings is solely controlled by the signals monitored and displayed by the device without providing any “diagnostic function” for analysis of the patient's biological condition and physiological state. For example, an increase in applied pressure is determined necessary when the patient's CO₂ differs from a value deemed normal by the prescribing physician. In this example, the device makes no diagnostic determination that the patient is hypo-ventilating or has undergone respiratory failure, the control is simply based on the sensor's measurement. The device will not manage the patient according to any incorporated diagnostic algorithm, rather it will adjust one or more prescription parameters to intervene to normalize the patient as prescribed by the clinician.

The system adjusting the parameter or parameters shall be constrained to approved ranges ascribed for the device and the class of patient. In other words, the clinician cannot intervene with a prescription that is not already allowed under the device's intended use or qualified range of settings. The procedures and techniques including the stability analysis, verification, and validation of the basic device will ensure compliance with the applicable safety standards and that all associated safety risks have been managed as far as possible in the design.

According to exemplary embodiments, the system and method for dynamically controlling the operation of a mechanical ventilator system includes accessing a prescription for physician managed control of the ventilator system wherein the prescription comprises an initial configuration for configuring the ventilator system to provide an initial mechanical ventilation prescription to the target person, a prescribed target biometric value of one sensor reading for a normal condition of the target person, a comparator formula by which the ventilator determines an ordinal relationship between the mean, median or most recent value of the biometric sensor during the titration interval and the prescribed target biometric value, and modification formulae by which the ventilator system generates a modification to a parameter to modify a current configuration of the ventilator system based on the ordinal relationship between the mean, median or most recent value of the biometric during the titration interval and the prescribed target biometric value and correlation between the ordinal relationship of the biometric and the modified parameter.

The method further comprises the steps dynamically controlling the operation of a mechanical ventilator system includes accessing a prescription for physician managed control of the ventilator system wherein the prescription comprises an initial configuration for configuring the ventilator system to provide an initial mechanical ventilation prescription to the target person, prescribed target biometric values for a plurality of sensor readings for a normal condition of the target person, a comparator formulae by which the ventilator determines the ordinal relationship between the mean, median or most recent values of the biometric sensors during the titration interval and the prescribed target biometric values, and modification formulae by which the ventilator system generates a modification to the parameters to modify a current configuration of the ventilator system based on the ordinal relationship between the mean, median or most recent values of the biometrics during the titration interval and the prescribed target biometric values and correlation between the ordinal relationship of the biometrics and the modified parameters.

According to some embodiments, the modification parameters are based on a correlation of the current measured biometric value to the parameter modified configuration of the ventilator system and a preprogrammed fixed increment or decrement value for the parameters. For example, the correlation between CO₂ and pressure support is negative and therefore a high reading of CO₂ will prompt an increase in pressure support by one unit of increment to encourage a decrease in CO₂. In other embodiments, the modification parameters are based on a correlation of the current measured biometric value to the parameter modified in the configuration of the ventilator system in proportion to the error between the measured biometric sensor reading and the target value. The modification is then a preprogrammed multiple of the fixed increment or decrement value of the parameters and the proportional factor of the difference between the biometric sensor reading the target value. For example, the correlation between CO₂ and Pressure support is negative and therefore a reading of CO₂ higher than the target by 5 mmHg will prompt an increase in pressure support by some increment to encourage a decrease CO₂ but the system will increase pressure support by twice that increment if the reading is higher than the target by 10 mmHg.

In some embodiments, the initial configuration may comprise one or more predetermined parameters and/or operating modes which include but are not limited to the following: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T).

The operational parameters associated with these modes include but are not limited to: Positive end expiratory pressure (PEEP), Pressure Support (PS), Respiratory rate (RR), Tidal volume (VT), Inspiratory airflow (V′), FiO2, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (ΔP). In many instances the base mode and intervention mode may be the same, but the intervention mode may include one of more differences in operation parameters that are all pre-determined by the prescribing clinician to affect positive outcomes when the patient is in need.

In some embodiments, the biometric sensor readings may include, but are not limited to the following: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

In some embodiments, biomarker calculators may include, but are note limited to: Peak inspiratory pressure (PIP), Peak pressure, Inspiratory time, Inspiratory-to-expiratory ratio, Time of pause, Trigger sensitivity, Support pressure, Expiratory trigger sensitivity, Plateau pressure (Pplat), Transpulmonary pressure, Transpulmonary driving pressure (ΔP), Mechanical energy, Mechanical power and intensity, and Pressure-time product per minute (PTP).

While embodiments of the invention have been described as having the features recited, it is understood that various combinations of such features are also encompassed by particular embodiments of the invention and that the scope of the invention is limited by the claims and not the description.

### BRIEF DESCRIPTION OF THE DRAWINGS

While the specification concludes with claims particularly pointing out and distinctly claiming particular embodiments of the instant invention, various embodiments of the invention can be more readily understood and appreciated from the following descriptions of various embodiments of the invention when read in conjunction with the accompanying drawings in which:

FIG. 1 illustrates a ventilator system;

FIG. 2 illustrates lungs and the inspiration and expiration flows in and out of the lungs;

FIG. 3 illustrates a schematic of a programmable physician managed targeting ventilation system in accordance with the teaching of the present invention;

FIG. 4 illustrates setting of a prescription parameter as a fixed value in a non-targeting state;

FIG. 5 illustrates targeted prescription operation of the system in with a single targeted parameter setting mode and showing activation of a targeted parameter prescription mode and selection of a prescription parameter,

FIG. 6 illustrates setting the targeted parameter, targeted value, interval units for update frequency, starting method, correlation signs and tolerance;

FIG. 7 illustrates a flow diagram for operation of the system in targeted parameter mode;

FIG. 8 illustrates a flow diagram for quick start operation of the system in targeted parameter mode or a system using proportional modification of parameters according to the error between the prescribed target and the measured monitored parameter; and

FIG. 9 is a chart of common ventilator operating modes and associated parameters set for each mode along with common values and units.

### DETAILED DESCRIPTION OF THE EXEMPLARY EMBODIMENTS

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal.

Some definitions and abbreviations that will be helpful for this application including the following:

Inspiratory flow refers to the flow of air entering into and flowing towards the lungs.

Expiratory flow refers to the flow of air exiting the lungs and flowing towards the glottis.

Rise Time is the rate at which the pressure ramps up to the prescribed or determined pressure level. The pressure generally rises during inspiration. The rise time of pressure can affect the flow rate and in particular the inspiratory flow rate and in more particular the peak inspiratory flow.

Fall Time is the rate at which the pressure ramps down to a determined pressure level. The pressure generally decreases during expiration. The fall time of pressure can affect the flow rate and in particular the expiratory flow rate and in more particular the peak expiratory flow.

Prescribed Pressure or Pressure Dosage is the amount of pressure that the ventilator ramps up to during use of the ventilator and is usually prescribed by a medical provider. Units of pressure are usually in the form of cm H₂O or centimeters of water column. Prescribed pressures generally range from 5-25 cm H₂O, and generally do not exceed 30 cm H₂O.

Mechanical ventilators are operational in several different modes. The most common modes of mechanical ventilation include but are not limited to: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T).

Volume control modes are generally favored for ventilation control, while pressure control modes are favored for assisted or spontaneously breathing patients. Both types of modes have advantages and disadvantages that are mainly related to the flow and pressure patterns of gas delivery.

Mechanical ventilators have many parameters that clinicians can adjust to treat a patient's condition, including but not limited to:

| Parameter | Value | Units |
|---|---|---|
| Peak Inspiratory Pressure | 0-99 | cm H2O, mbar, hPa |
| Peak Inspiratory Flow | 0-200 | L/min |
| Inhaled Tidal Volume | 0-2000 | ml |
| Exhaled Tidal Volume | 0-2000 | ml |
| Respiratory Rate | 0-120 | BPM |
| Minute Volume | 0-40 | L/min |
| % Spontaneous Trigger | 0-100 | % |
| Asynchrony Index | 0-100 | % |
| Lung Compliance | 1-200 | ml/cm H2O, ml/mbar, |
| | | ml/hPa |
| Static Lung Compliance** | 1-200 | ml/cm H2O, ml/mbar, |
| | | ml/hPa |
| Static Lung Resistance** | 1-200 | cmH2O/Lps, mbar/Lps, |
| | | hPa, Lps |
| Plateau Pressure** | 0-99 | cm H2O, mbar, hPa |
| Alveolar Pressure | 0-99 | cm H2O, mbar, hPa |
| Expiratory Resistance | 1-200 | cmH2O/Lps, mbar/Lps, |
| | | hPa, Lps |
| Inspiratory Resistance | 1-200 | cmH2O/Lps, mbar/Lps, |
| | | hPa, Lps |
| iPEEP | 0-40 | cm H2O, mbar, hPa |
| FiO2 | 21-100 | % |
| SpO2 | 5-100 | % |
| Heart Rate | 18-321 | BPM |
| ETCO2 | 5-150 | mmHg |
| TcCO2 | 5-150 | mmHg |
| Peak Expiratory Flow | 0-200 | L/min |
| I:E Ratio | 2:1:1:99 | |
| Expiratory Time | 1-60 | sec |
| RSBI | 8-999 | Breaths/min/L |
| Vt/kg IBW | 1-30 | cc/KgIBW |
| Flow Bias Ratio* | 10:1:1:10 | Insp:Exp |
| Average Leak | 0-200 | L/min |
| Leak Ratio to Normal | 0.0:10 | None |
| AHI (Apnea Hypopnea Idx) | 0-300 | per hour |
| Expiratory Time Constant | 0.1-10 | sec |
| Resp Rate-Oxygenation (ROX) | 0-99 | points |

Abbreviations associated with the above are set forth below.

| Parameter Name | Abbreviation |
|---|---|
| Peak Inspiratory Pressure | PIP |
| Peak Inspiratory Flow | PIF |
| Tidal Volume | Vt |
| Respiratory Rate | RR |
| Minute Volume | MVexp |
| % Spontaneous Trigger | % Spon Trigger |
| Asynchrony Index | Asynch Idx |
| Lung Compliance | Clung |
| Static Lung Compliance** | Cstat |
| Static Lung Resistance** | Rstat |
| Plateau Pressure** | Pplat |
| Alveolar Pressure | Palv |
| Expiratory Resistance | Rexp |
| Inspiratory Resistance | Rinsp |
| Intrinsic PEEP | iPEEP |
| Fraction of Inhaled O2 | FiO2 |
| Saturation of Peripheral O2 | SpO2 |
| Heart Rate | HR |
| End Tidal Carbon Dioxide | ETCO2 |
| Transcutaneous Carbon Dioxide | TcCO2 |
| Peak Expiratory Flow | PEF |
| Inspiratory Expiration Ratio | I:E Ratio |
| Rapid Shallow Breathing Index | RSBI |
| Tidal Volume per Ideal Body Weight | Vt/kg IBW |
| Flow Bias Ratio* | PEF/PIF Ratio |
| Average Leak | Ave Leak |
| Leak Ratio to Normal | Leak Ratio |
| Apnea Hypopnea Index | AHI |
| Expiratory Time Constant | TCexp |
| Inhaled Tidal Volume | Vti |
| Exhaled Tidal Volume | Vte |
| Resp Rate-Oxygenation (ROX) | ROX |

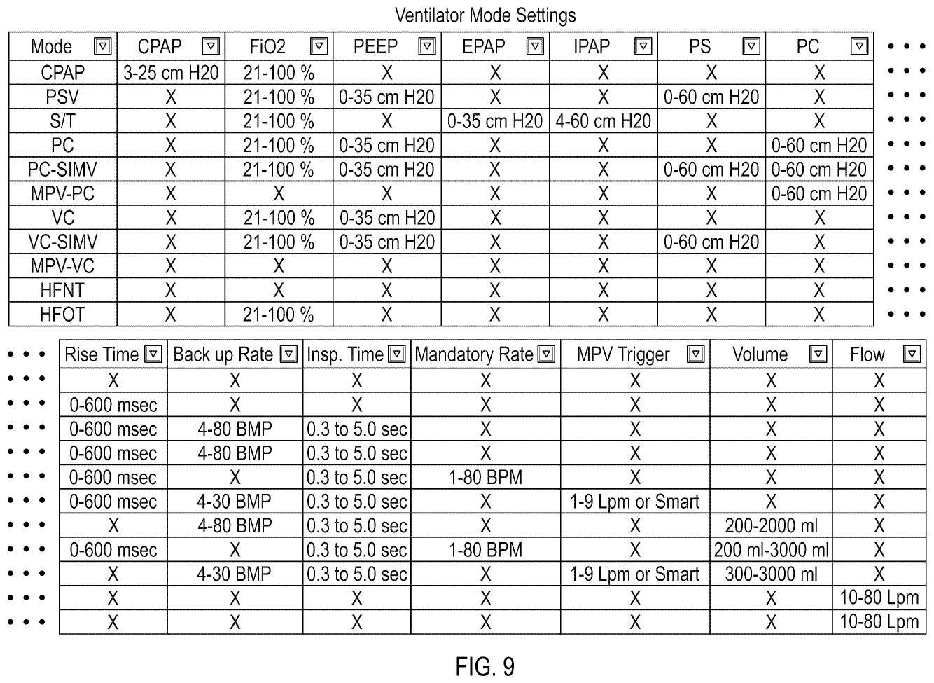

Finally, parameters to be set depend on modes. An exemplary setting chart is set forth in FIG. 9, where the settings names are the column headers.

Additional definitions are also set forth below.
Positive end-expiratory pressure (PEEP)
Inspiratory airflow (V′)
Percentage of inspired oxygen (FiO₂): Normal is 35-50%, but can be increased to 75-100% if needed
Air pressure (SetP)
Peak inspiratory pressure (PIP): The sum of PEEP and SetP
Peak pressure

Inspiratory time: Inspiratory Time is the length of time during which the ventilator delivers the inspiratory pressure.
Inspiratory-to-expiratory ratio
Time of pause
Trigger sensitivity
Support pressure
Expiratory trigger sensitivity

Ventilators can also produce ventilator-derived parameters, i.e. biomarker calculators, which can be used to guide ventilatory strategies and detect problems with the ventilator or changes in the patient:

Intrinsic PEEP (PEEPi): The residual pressure when the expiratory phase isn't fully completed
Plateau pressure (Pplat): Equal to alveolar pressure when airflow is zero
Transpulmonary pressure
Transpulmonary driving pressure (ΔP)
Mechanical energy
Mechanical power and intensity
Pressure-time product per minute (PTP)

Patients on mechanical ventilation are usually monitored in an intensive care unit (ICU) and have monitors that measure several values related to respiration, including but not limited to: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

As will be described in more detail hereinbelow, the invention provides a novel, programmable ventilator system and methodology where the clinician may prescribe and individualize the selection and monitoring of a targeted operational or outcome parameter, or patient biometric sensor value (target condition value), and the ventilator system's automatic response when that targeted parameter value and is not equivalent to the prescribed or “targeted” value +/− tolerance. The prescribing clinician may define a single prescribed target value or a combination of prescribed target value corresponding to any biometric sensor measurements and/or any measured corresponding device parameters deviating from targeted values to define the dynamic control of the ventilator system automatically to normalize the patient.

In a first embodiment, the system is programmed with an initial “therapeutic” configuration and/or mode having a set operational mode and/or parameters. For example, in the base therapy state, the patient may be receiving Continuous Positive Airway Pressure (CPAP) with particular operational parameters, such as pressure and FiO₂.

In accordance with the teachings of the present invention the system is further programmed with a managed configuration parameter using targeting, target parameter sensor and the patient's target value for the sensor reading, interval frequency, correlation signs, and tolerance. For example, the target biometric sensor may be a pulse oximeter connected to the patient measuring peripheral oxygen concentration. (SpO2), the targeting parameter is FiO₂. In this example, the correlation between SpO2 and FiO2 is positive. The interval may be prescribed as 15 minutes with a tolerance of 1%. In other words, if the measurement of SpO₂ is more than 1% lower than the target SpO2, the FiO2 will be adjusted by one increment higher in 15 minute intervals to encourage an increase in SpO2 until SpO2 reaches the target value or the FiO2 reaches the allowable upper limit of FiO2.

The present invention overcomes the issues of inappropriate response time incurred due to remote monitoring and more specifically the issue of “no response” when caregivers are absent, or the patient is unable to intervene on his own.

There are no underlying algorithms contained within the device programming to make clinical assessments of the patient, and the device does not decide what an appropriate response could be. Biometric and operating conditions are measured, target values are set, and a prescribed response interval is set based on the judgment of the physician attending to the specific patient who is treated. The system will simply only operate according to the direction of the programming clinician. There is full transparency of how, when, and why a setting will change.

An important distinction with Programmable Ventilation with targeting according to the present disclosure, is that modification or adjustment of the ventilator's operational parameters are solely controlled by the heath data signals that are already monitored and displayed by the device without providing any “diagnostic function” for analysis of the patient's biological condition and physiological state. The actions taken by the system are done automatically only according to the prescription of the expert clinician.

The control system adjusting the parameter or parameters is constrained to approved ranges ascribed and programmed for the device and the class of patient. In other words, the targeting system will not adjust an operational parameter to a value that is not already allowed under the device's intended use or qualified range of settings (saved prescription). The procedures and techniques including the stability analysis, verification, validation will ensure compliance with the applicable safety standards and that all associated safety risks have been managed as far as possible in the design.

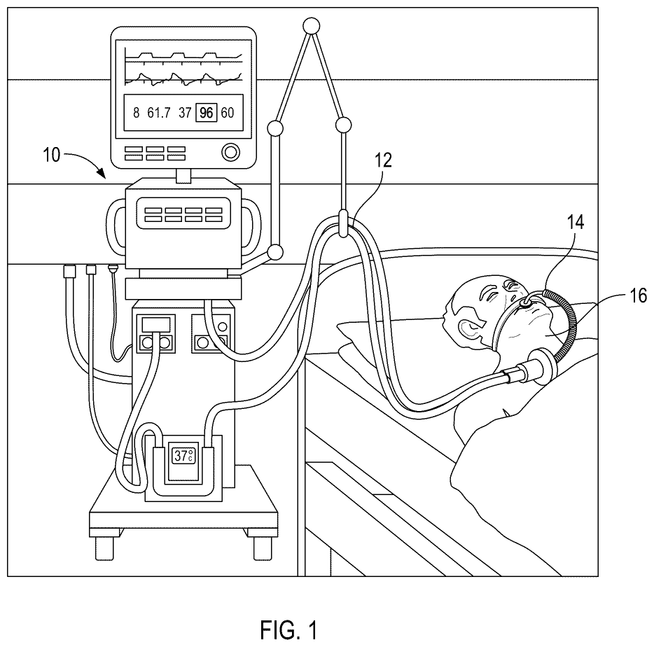

Turning now to the drawing figures, FIG. 1 illustrates a basic ventilator system 10 that provides pressurized air through the tube 12 into an airway adaptor 14, such as a mask, to the user/patient 16. In some instances, a mask is not used, where the tube is directly fed into the trachea, such as a tracheostomy.

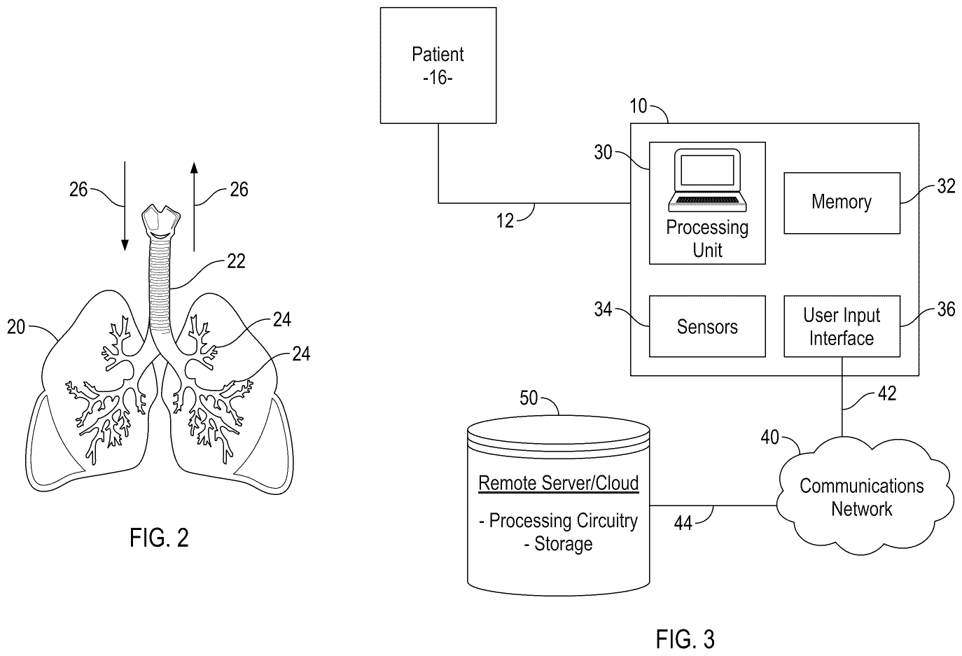

FIG. 2 illustrates lungs 20, including the trachea 22 and the bronchi of the lungs 24. The inspiration flow path 26 travels into the trachea 22 and into bronchi 24, whereas the expiration flow path 28 travels or flows out from the lungs 20 and bronchi 24 into and out of the trachea 22.

FIG. 3 illustrates a schematic of a targeting mechanical ventilation system 100 that includes a ventilator system 10, which includes a processing unit 30 configured to receive input operating parameters (as set forth hereinabove), via a user/patient input interface 36, including targeting prescription selections and parameters, as well as direct and processed sensor data captured by sensors 34, to recall and place data into memory 32, and direct communications over a network 40 to remote server/cloud 50 that also includes processing circuitry and storage. Directed communications 42 can be made to and from the communications network, which can make directed communications 44 to and from the remote server/cloud 50. Cloud computing is generally understood in the art to mean the delivery of computing services-including servers, storage, databases, networking, software, analytics, and intelligence-over the Internet (“the cloud”) to offer faster innovation, flexible resources, and economies of scale.

According to exemplary embodiments of the invention, a method for dynamically controlling the operation of a ventilator system includes accessing a saved prescription for automatic control of the ventilator system wherein the prescription includes the steps of:
accessing a prescription for automatic control of the ventilator system in providing mechanical ventilation to the target person, the prescription comprising:
   an initial configuration for configuring the ventilator system to provide an initial mechanical ventilation to the target person,
   a prescribed target biometric value for a target condition of the target person,
   comparator formulae by which the ventilator determined an ordinal relationship of the current condition value to the prescribed target biometric value; and
   modification formulae by which the ventilator system generates modification parameters to modify a current configuration of the ventilator system based on the ordinal relationship between the current condition value and the prescribed target biometric value.

The method further comprises the steps of:
configuring the ventilator system according to the initial configuration;
operating the ventilator system according to the initial configuration;
during operation of the ventilator system, continuously monitoring readings from a plurality of biometric sensors and biomarker calculators corresponding to the target person and:
   at predetermined titration intervals, determining a current condition value for the target person based one or more readings from said plurality of biometric sensors and biomarker calculators;
   determining whether the current condition value of the target condition meets the target biometric value according to the comparator formulae; and
   upon determining that the current condition value does not meet the target biometric value;
   generating modification parameters to modify the current configuration of the ventilator system based on the ordinal relationship between the current condition value and the prescribed target biometric value; and
   reconfiguring the current configuration of the ventilator system according to the modification parameters.

The modification condition is not the machine's clinical assessment that the initial parameter setting is no longer optimal, but that combination of respiratory measurements which the clinician has deemed the modification condition describing when the patient requires a modification to encourage the target score, and or when the patient no longer requires the modification because the target score is within an acceptable tolerance.

In some embodiments, the modification parameters may be further based on a correlation of the current condition score to the configuration of the ventilator system.

In some embodiments, the initial configuration may comprise one or more predetermined operational parameters and/or operating modes which include but are not limited to the following: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T).

The operational parameters associated with these modes include but are not limited to: Positive end expiratory pressure (PEEP), Pressure Support (PS), Respiratory rate (RR), Tidal volume (VT), Inspiratory airflow (V′), FiO2, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (ΔP) and combinations of the modes noted and the operational parameters set forth above.

In some embodiments, the biometric sensor readings may include, but are not limited to the following: heart rate, respiratory rate, blood pressure, Oxygen Saturation (SpO₂) and End-Tidal Carbon Dioxide (ETCO₂).

In some embodiments, the biomarker calculators are selected from the group consisting of: Peak inspiratory pressure (PIP), Peak pressure, Inspiratory time, Inspiratory-to-expiratory ratio, Time of pause, Trigger sensitivity, Support pressure, Expiratory trigger sensitivity, Plateau pressure (Pplat), Transpulmonary pressure, Transpulmonary driving pressure (ΔP), Mechanical energy, Mechanical power and intensity, and Pressure-time product per minute (PTP). Calculation of the noted biomarkers is generally known in the art and will not be further described.

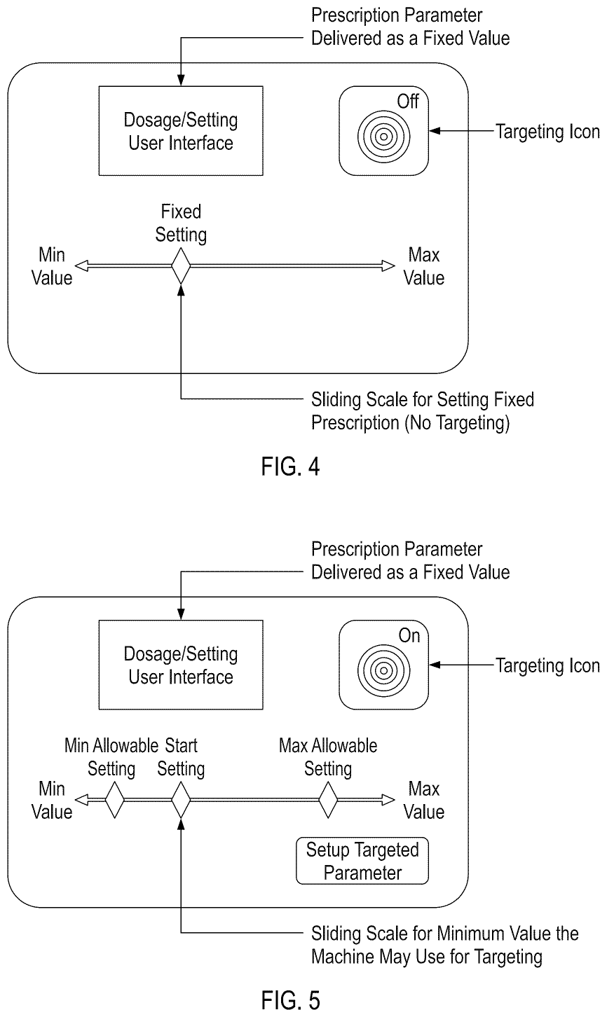

Referring now to FIGS. 4-7 a first exemplary configuration of the system and methodology are illustrated and described.

Setting up the Targeting Program

It is typical to set a prescription in mechanical ventilation with settings associated with that prescription. For example, for CPAP prescriptions the pressure and fraction of inhaled oxygen (FiO2) are set. For Volume Control ventilation, the volume, inspiratory time, back up rate and FiO2 are set. Usually, these settings are titrated in the clinical settings at discharge and then a chronic patient is sent home with this titration.

FIG. 4 illustrates the clinician's selection of providing a fixed setting without targeting. A targeting icon is toggled OFF and a fixed target value is set between a minimum and maximum range.

In the present invention, the user has the option to choose a range of settings within the approved range of the device, that will be adjusted based on the rules of the targeting system supplied to the machine by the user.

Turning to FIG. 5, the targeting system is enabled (icon toggled to ON), and the user decides (prescribes) the guard rails within the machine's minimum and maximum values through which the system will decide which value to deliver.

The settings include:
1. a minimum setting for the patient that is greater than or equal to the minimum setting approved in the specification of the device for the therapy and patient demographic;
2. a maximum setting for the patient that is less than or equal to the maximum setting approved in the specification of the device for the therapy and patient demographic; and
3. a starting setting that is inclusively between the minimum and maximum setting above.

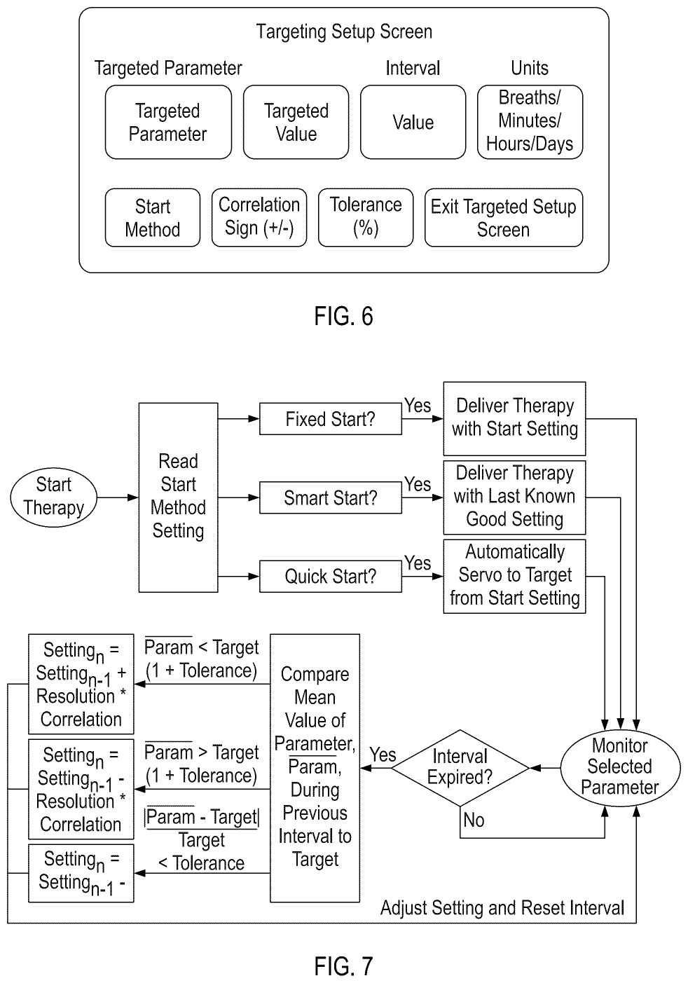

When targeting is enabled, the targeting system must be setup with a number of user selections which are programmed through a series of additional icons (see FIG. 6).

The selections for the targeting system are shown and explained the setting Table below.

| Setting | Description |
|---|---|
| Targeted | A parameter selected from a list of biometric sensors and biomarker |
| Parameter | calculators within the ventilator or connected devices for which the |
| | clinician has a target value indicating normalcy |
| Targeted Value | The desired quantity for the targeted parameter |
| Titration | A quantity in time that when combined with units tells the machine how |
| Interval | frequently to update the prescription setting used to target a parameter |
| Units | A unit of time or number of breaths that when combined with the Interval |
| | Value determines how frequently to update the prescription setting used to |
| | target a parameter. (Can be breaths, seconds, minutes, hours, days, etc.) |
| Start Method | An input from the clinician to tell the machine how to initiate therapy. |
| | The choices can be 1. Start at a fixed value 2. Start smart (where the |
| | machine left off from a previous therapy session) 3. Initiate a temporary |
| | servo to rapidly converge to the target. |
| Correlation Sign | A positive one (+1) or a negative one (−1) that indicates the correlation |
| | between the setting and the monitored parameter. For example, FiO2 |
| | (Setting) and SpO2 (parameter) are positively correlated (+1) because |
| | increasing FiO2 will generally increase SpO2. Alternatively, Volume |
| | (Setting) and Respiratory Rate (parameter) are negatively correlated (−1) |
| | because increasing Volume will generally decrease respiratory rate. |
| Tolerance | A percentage between 0 and 1, that tells the machine at what accuracy |
| | relative to the target value, should the targeting system cease to adjust |
| | therapy. For example, if the user selects a target volume of 500 ml, with |
| | a tolerance of 10%, the machine will cease to adjust therapy if the |
| | measurement of volume is between 450 and 550 ml. +/−10% around 500 ml. |

Once initiated the system operates according to the flow diagram as illustrated in FIG. 7 delivering the initial ventilation configuration according to the selected target parameter and target settings prescribed.

The system memory 32 may allow more than one set of prescriptions to be stored along with respective target parameters conditions.

In the present embodiment, a targeted parameter value is introduced and defined that does not require an expert to manually detect the condition and the need to manually change the prescription and operating parameters.

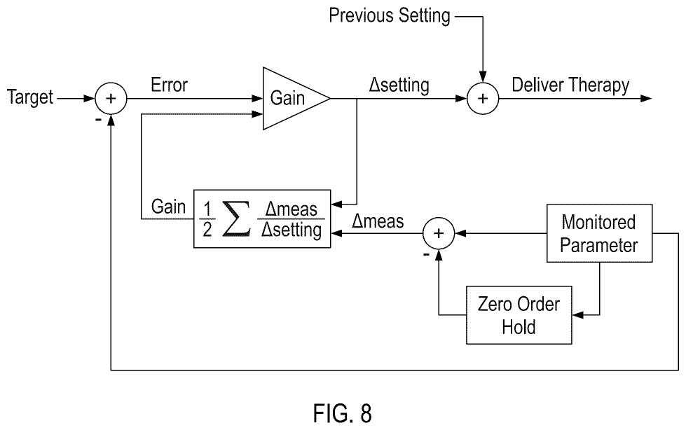

Turning to FIG. 8, a flow diagram for an alternative starting mode (Quick Start) is illustrated. This flow diagram also describes the use of proportional targeting described above.

If initiated in Quick Start mode, the system may quickly adjusts to the target parameter from the initial Start settings because it is not limited to the smallest increment or decrement of the configured parameter.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A method for dynamically controlling the operation of a ventilator system in providing mechanical ventilation to a target person, comprising:
accessing a prescription for automatic control of the ventilator system in providing mechanical ventilation to the target person, the prescription comprising:
an initial configuration for configuring the ventilator system to provide an initial mechanical ventilation to the target person,
a prescribed target biometric value for a target condition of the target person,
comparator formulae by which the ventilator determines an ordinal relationship of a current condition value to the prescribed target biometric value; and
modification formulae by which the ventilator system generates modification parameters to modify a current configuration of the ventilator system based on the ordinal relationship between the current condition value and the prescribed target biometric value;
configuring the ventilator system according to the initial configuration;
operating the ventilator system according to the initial configuration;
during operation of the ventilator system, continuously monitoring readings from a plurality of biometric sensors and biomarker calculators corresponding to the target person and periodically:
at predetermined titration intervals, determining a current condition value for the target person based one or more readings from said plurality of biometric sensors and biomarker calculators;
determining whether the current condition value of the target condition meets the target biometric value according to the comparator formulae; and
upon determining that the current condition value does not meet the target biometric value;
generating modification parameters to modify the current configuration of the ventilator system based on the ordinal relationship between the current condition value and the prescribed target biometric value; and
reconfiguring the current configuration of the ventilator system according to the modification parameters.

2. The method of claim 1, wherein the modification parameters are further based on a correlation of the current condition value to the configuration of the ventilator system.

3. The method of claim 1, wherein the initial configuration comprises one or more predetermined parameters and/or operating modes selected from the group consisting of: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T); Positive end expiratory pressure (PEEP), Pressure Support (PS), Respiratory rate (RR), Tidal volume (VT), Inspiratory airflow (V′), FiO₂, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (ΔP) and combinations thereof.

4. The method of claim 2, wherein the initial configuration comprises one or more predetermined parameters and/or operating modes selected from the group consisting of: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T); Positive end expiratory pressure (PEEP), Pressure Support (PS), Respiratory rate (RR), Tidal volume (VT), Inspiratory airflow (V′), FiO₂, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (ΔP) and combinations thereof.

5. The method of claim 1 wherein the sensor readings comprise readings selected from the group consisting of: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

6. The method of claim 2 wherein the sensor readings comprise readings selected from the group consisting of: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

7. The method of claim 3 wherein the sensor readings comprise readings selected from the group consisting of: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

8. The method of claim 4 wherein the sensor readings comprise readings selected from the group consisting of: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

9. The method of claim 1 wherein said biomarker calculators are selected from the group consisting of: Peak inspiratory pressure (PIP), Peak pressure, Inspiratory time, Inspiratory-to-expiratory ratio, Time of pause, Trigger sensitivity, Support pressure, Expiratory trigger sensitivity, Plateau pressure (Pplat), Transpulmonary pressure, Transpulmonary driving pressure (ΔP), Mechanical energy, Mechanical power and intensity, and Pressure-time product per minute (PTP).

10. The method of claim 2 wherein said biomarker calculators are selected from the group consisting of: Peak inspiratory pressure (PIP), Peak pressure, Inspiratory time, Inspiratory-to-expiratory ratio, Time of pause, Trigger sensitivity, Support pressure, Expiratory trigger sensitivity, Plateau pressure (Pplat), Transpulmonary pressure, Transpulmonary driving pressure (ΔP), Mechanical energy, Mechanical power and intensity, and Pressure-time product per minute (PTP).

11. The method of claim 3 wherein said biomarker calculators are selected from the group consisting of: Peak inspiratory pressure (PIP), Peak pressure, Inspiratory time, Inspiratory-to-expiratory ratio, Time of pause, Trigger sensitivity, Support pressure, Expiratory trigger sensitivity, Plateau pressure (Pplat), Transpulmonary pressure, Transpulmonary driving pressure (ΔP), Mechanical energy, Mechanical power and intensity, and Pressure-time product per minute (PTP).

12. The method of claim 4 wherein said biomarker calculators are selected from the group consisting of: Peak inspiratory pressure (PIP), Peak pressure, Inspiratory time, Inspiratory-to-expiratory ratio, Time of pause, Trigger sensitivity, Support pressure, Expiratory trigger sensitivity, Plateau pressure (Pplat), Transpulmonary pressure, Transpulmonary driving pressure (ΔP), Mechanical energy, Mechanical power and intensity, and Pressure-time product per minute (PTP).

13. The method of claim 1 wherein the current condition value comprises a mean, median or most recent value of a predetermined biometric sensor.

14. The method of claim 1 wherein the current condition value comprises a plurality of current condition values, and the comparator formulae determines ordinal relationships between the current condition values and the prescribed target biometric values.

15. A dynamically configurable ventilator system for providing mechanical ventilation to a target person according to a prescription, configured to operate in accordance with the method of claim 1.
